(19) United States
(12) Patent Application Publication
(10) Pub. No.: US 2008/0276971 A1
(43) Pub. Date: Nov. 13, 2008

(54) Contact Lens Cleaning Devices

(76) Inventor: Charles Ifejika, London (GB)

Correspondence Address: Theresa Fritz Camoriano;Camoriano and Associates, 8225 Shelbyville Road, Louisville, KY, 40222, US

(21) Appl. No.: 10/568,493

(22) PCT Filed: Aug. 13, 2004
(86) PCT No.: PCT/GB2004/003517

§ 371 Date: Aug. 1, 2008

(30) Foreign Application Priority Data
Aug. 18, 2003 (GB) 0319356.2

(51) Int. Cl. G02C 13/00; A61L 12/02

(52) U.S. Cl. 134/32; 134/184; 134/140

(57) ABSTRACT

A device for cleaning contact lenses comprising a solution chamber and a motor (4) adapted to impart a reciprocating and vibrational motion to a contact lens holder (3). The contact lens holder (3) is adapted to receive one or more contact lenses, which holder, in use, is for insertion in the solution chamber (2), the holder (3) being adapted to permit fluid flow to the contact lens or lenses. The holder has first and second baskets (34,36) each adapted to receive a contact lens, which lens has an inner and an outer surface, the first and second baskets being oriented such that, in use, the respective inner surfaces of the contact lenses are remote from one another. In use, the reciprocating and vibrational motion imparted to the contact lens holder (3) generates bubbles in a solution in the solution chamber (2), which bubbles contribute to fluid turbulence at a surface of the contact lens, thereby assisting in the cleaning of the lens.

The invention relates to a contact lens cleaning device and a method for cleaning contact lenses

A particular problem for contact lens wearers is microbial contamination. Infections due to microbial keratitis, acanthamoeba and ulcerative keratitis are recurring problems associated with contact lens wear. The problems may arise, for example, when a contact lens is not cleaned sufficiently by the lens wearer and the bacterial load of the lens increases such that a biofilm forms on the lens. In such cases not all lens cleaning solutions may be strong enough to kill residual bacteria. Similarly the contact lens may harbour infectious organism such as acanthamoeba, which can also contaminate the lens case in addition to the lens resulting in time in a devastating keratitis.

Most known lens care systems do not provide for ease of use. Typically a user is required to clean their lenses for 10-30 seconds with their fingers with a cleaning solution before rinsing and disinfecting the lens, which may take up to six hours. Often the lens will need to be rinsed again prior to use. Due to the labour intensive nature of the process it is well established that a significant number of contact lens wearers omit one or more steps, leading to complications and infection.

A number of electromechanical devices have been proposed to assist in the cleaning of contact lenses but few devices have been able to establish themselves commercially as either they do not work efficiently enough to persuade users to switch or they are simply too expensive or cumbersome to be practical.

EP0394254 discloses apparatus comprising an agitator with a reciprocating motion, which apparatus is adapted to clean the lens in a short time with minimal effort. In common with other devices, the illustrated device suffers from the problems that it is not particularly efficient at cleaning in the interior of the lens surface, in particular achieving sufficient cleaning of acanthamoeba infection, which is where the most serious problems with contamination occur and also suffers from high manufacturing costs.

The present invention seeks to provide a contact lens cleaning device which is adapted to clean both sides of a contact lens.

According to the invention there is provided a device for cleaning contact lenses comprising a solution chamber and a motor and a contact lens holder adapted to receive one or more contact lenses, which holder, in use, is for insertion in the solution chamber, the holder being adapted to permit fluid flow to the contact lens or lenses, wherein the apparatus further comprises means adapted to impart a reciprocating and vibrational motion to the contact lens holder characterised in that the lens holder (3) comprises first and second baskets (34,36) each adapted to receive a contact lens, which lens has an inner and an outer surface, the first and second baskets being oriented such that, in use, the respective inner surfaces of the contact lenses are remote from one another, wherein, in use, the means adapted to impart a reciprocating and vibrational motion to the contact lens holder (3) generates bubbles in a solution in the solution chamber (2), which bubbles contribute to fluid turbulence at a surface of the contact lens, thereby assisting in the cleaning of the lens.

Preferably, the reciprocating and vibrational motion operates in a frequency range of 10 to 100 Hz, more preferably 45 to 55 Hz. Preferably, the means adapted to impart the reciprocating and vibrational motion comprises a motor, which motor imparts a drive to an offset pin, which pin is drivingly engaged in a slot of a support arm, which support arm is positively connected to the lens holder, such that rotation of the off-set pin imparts said reciprocating and vibrational motion.

According to a second aspect of the invention, there is provided a method for cleaning contact lenses comprising inserting a contact lens holder in a solution chamber, which solution chamber contains a cleaning liquid, the holder being adapted to permit fluid flow to the contact lens or lenses, applying a simultaneous reciprocating and vibrational motion to the lens holder characterised in that the lens holder is provided with first and second baskets each adapted to receive a contact lens, which lens has an inner and an outer surface, the first and second baskets being oriented such that, in use, the respective inner surfaces of the contact lenses are remote from one another, wherein the reciprocating and vibrational motion generates bubbles in the cleaning liquid, which bubbles contribute to fluid turbulence at a surface of the contact lens, thereby assisting in the cleaning of the lens.

Preferably, the cleaning solution has at least ten bubbles per 5 ml of liquid at any one time. Preferably, the cleaning liquid is a contact lens cleaning fluid or saline solution. Preferably, the reciprocating-vibrational motion is applied for one minute, the device is turned off, and then the reciprocating-vibrational motion is applied for a further minute.

Contact lenses are usually stored in cases, which have lenses that are held within a contact lens holder. The holder has baskets, which cradle the contact lenses with the convex side of the lens facing inward. The contact lenses rest upon a concave plastic or rubber surface, which closely matches the inner surface of the lens. This is the industry standard and there is no requirement for any change to this configuration. However, if lenses are held in this way the part of the lens, which requires the most thorough cleaning, is facing inward and towards each other. Therefore when an outside cleaning agent acts to clean the lenses, the surfaces requiring the most thorough cleaning are hidden from the immediate, most significant cleaning action of the outside agent. The outside cleaning agent will push the lenses onto the plastic or rubber surface upon which the lens rests. This means that the inner surface of the lens, which may have deposits and or bacteria, will be pushed onto the surface upon which the lens rests. The deposits and or bacteria can contaminate the surface upon which the lens rests and the inward turned lenses now act as a barrier to the outside cleaning source, preventing it from cleaning the surface upon which the lens rests or the inner surface of the contact lens.

The invention advantageously solves this problem in that, the inner surface of the contact lens is in use oriented such that it faces away from the inner surface of an opposed contact lens, thereby obviating the problem inherent in the industry standard design.

The invention also advantageously provides for a constant generation of bubbles in the cleaning liquid during use. The bubbles tend to nucleate at the surface of the lens and generate a fluid turbulence there, which acts to in effect scrub the lens thereby enhancing the cleaning effect of the agitation.

An exemplary embodiment of the invention will now be described in greater detail with reference to the drawing in which:

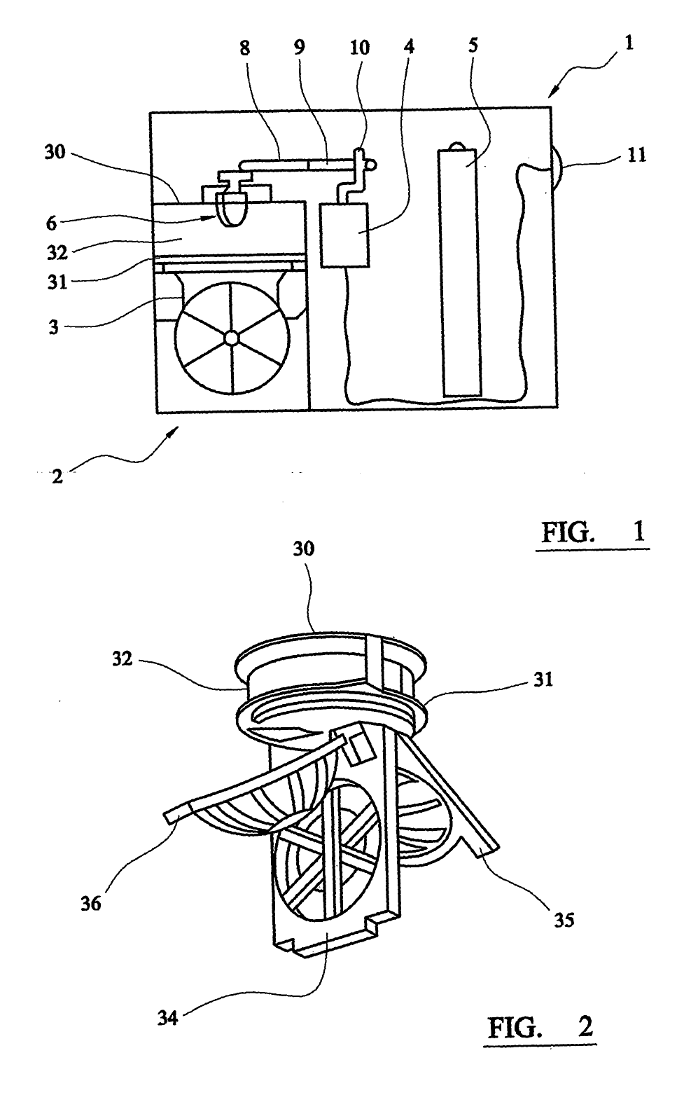

FIG. 1 shows a contact lens cleaning device

The contact lens cleaning device comprises a housing 1, a solution chamber 2 and a contact lens holder 3. The housing 1 contains a drive mechanism adapted to impart a simultaneous reciprocating-vibratory motion to the lens holder 3.

The drive mechanism comprises a motor 4, control electronics and switch 11 together with a power supply such as batteries 5, which are located in the housing 1 to drive the motor 4. The contact lens holder 3 is, in use, drivingly connected to the motor by means of two curved shaped key parts 6, which positively connect with the upper part of the lens holder 3. The key parts 6 comprise two arms of a unitary connection with is drivingly connected to the motor such that agitation can be imparted to the lens holder 3. The key parts 6 are held with some play between two bearing surfaces in the housing 1. The connection has a further arm 8 having a slot 9 adapted to receive an off-set pin 10 and the slotted part of the connecting arm is provided with greater play than the key part 6. The offset pin 10 is drivingly connected to the motor 4. A switch 11 actuates the motor 4, which in use rotates the offset pin 10 to impart a reciprocating motion to the key parts 6 and hence the lens holder 2. A further vibratory component is provided by the difference in play provided.

This has proved to be a particularly effective form of agitation for cleaning the contact lens. The motor imparts this form of motion at a frequency of between 10 and 100 Hz and in general a frequency of around 45-55 Hz is preferred.

FIG. 2 shows the lens holder 3 in greater detail, which lens holder 3 comprises a mounting section 30 adapted to positively connect with the key parts 6. The mounting section 30 is held with some play in a piston 32 which is provided with an O-ring 31 to provide a seal against the solution chamber wall. The end of the mounting piece remote from the connection 30 is provided with a three part lens holder adapted to receive two contact lenses.

The lens holder comprises a fixed central member 34 of generally circular diameter having eight spokes to permit fluid flow through the member. Two baskets 35,36 having a similar generally circular diameter with eight spokes attached to a central boss are pivotally connected to the base of the central member 34, so that they can open out to receive the contact lens and then pivot back to mate with the central member 36 to prevent the lens from being dislodged when the agitation is applied to the lens holder. The baskets are shaped to receive the curved contact lens and can have two orientations, namely where the inner surface of the lens faces outwards during cleaning or where with the inner surface of the lens faces inwards during cleaning. In known systems the inner surface is usually oriented inwardly.

In most cases the greatest deposits are to be found on the inner surface of the contact lens, which is also the surface where there is the greatest risk of bacterial transmission to the eye.

In use, a contact lens is placed in each respective basket 35,36 and these are brought to the closed position adjacent the central member 34. A cleaning solution is then placed in the solution chamber 3. The cleaning solution can be a conventional contact lens cleaning solution but it is also possible to use sterile solutions such as saline solution to achieve a satisfactory cleaning of the lens. If a typical contact lens diameter of 14.2 mm is used, the solution chamber 2 will have an approximate volume of 10 ml. As the contact lenses and holder have a displacement volume, typically 5 ml of solution will be required to immerse the contact lenses, leaving a small amount of air in the chamber between the piston 32 and the surface of the liquid.

In use, the motor is actuated and the combined vibratory-reciprocating motion is applied to the lens holder 3 in the solution in solution chamber 2. Due to the action of the lens holder the pressure at the centre of the solution chamber is slightly higher than at the edge of the chamber, which results in an exaggeration of the meniscus. Additionally, the action of the lens holder 2 results in turbulence in the fluid, which in turn encourages the generation of air bubbles as the air in the chamber is drawn into the solution.

The generation of air bubbles results in a vortex of bubbles, which typically are attracted towards the surface of the lens and the lens holder as these provide the nucleation sites. The air bubbles therefore tend to circulate around the chamber from the high pressure region in the centre downwards and then to rise up the outside of the chamber where the pressure is lower. The air bubbles themselves appear to have a chaotic motion across the surface of the contact lens, in particular across the surface of the lens remote from the central member 34. The chaotic motion of the bubbles across the surface causes a chaotic fluid flow across the surface of the lens, which acts to scrub the lens clean, in particular the inner surface of the lens. The combined reciprocating and vibrational motion causes a constant generation of bubbles with a typical minimum of ten bubbles in 5 ml of fluid at any one time. The rate of generation of bubbles is dependent on the fluid used and also the frequency of the reciprocating vibration motion and can be significantly higher with several tens of bubbles being present at any one time.

If a standard lens cleaning solution is used as a cleaning agent and the device is actuated for two separate one minute periods, the device will produce a 5 log removal (99.999%) of acanthamoeba trophozoites and cysts; pseudomonas aeruginosa; staphyloccus aureus, serratia marcescens; candida albicans and fusarium solani. No prior art devices are known to achieve this.

Although the device has been described as having the inner surface of the lens facing outwards, it would also be possible to have the inner surface of the lens facing inwards. It would also be possible to use other drive mechanisms other than the offset pin arrangement described such as an eccentric and pushrod or various solenoid arrangements. Although the invention as described relates to cleaning contact lenses, it could also be used for cleaning other items such as small electrical components or scientific equipment.

1. A device for cleaning contact lenses comprising a solution chamber (2) and a contact lens holder (3) adapted to receive one or more contact lenses, which holder, in use, is for insertion in the solution chamber (2), the holder (3) being adapted to permit fluid flow to the contact lens or lenses, wherein the apparatus further comprises means adapted to impart a reciprocating and vibrational motion to the contact lens holder (3) characterized in that the lens holder (3) comprises first and second baskets (34,36) each adapted to receive a contact lens, which lens has an inner and an outer surface, the first and second baskets being oriented such that, in use, the respective inner surfaces of the contact lenses are remote from one another, wherein in use, the means adapted to impart a reciprocating and vibrational motion to the contact lens holder (3) generates bubbles in a solution in the solution chamber (2), which bubbles contribute to fluid turbulence at a surface of the contact lens, thereby assisting in the cleaning of the lens.

2. A device according to claim 1, wherein the reciprocating and vibrational motion operates in a frequency range of 10 to 100 Hz.

3. A device according to claim 1, wherein the means adapted to impart the reciprocating and vibrational motion comprises a motor (4), which motor (4) imparts a drive to an offset pin (10), which pin (10) is drivingly engaged in a slot (9) of a support arm (8), which support arm is positively connected to the lens holder (3), such that rotation of the off-set pin imparts said reciprocating and vibrational motion.

4. A device according to claim 2, wherein the frequency is 45 to 55 Hz.

5. A method for cleaning contact lenses comprising inserting a contact lens holder (3) in a solution chamber (2), which solution chamber contains a cleaning liquid, the holder (3) being adapted to permit fluid flow to the contact lens or lenses, applying a simultaneous reciprocating and vibrational motion to the lens holder (3) characterized in that the lens holder is provided with first and second baskets (34,36) each adapted to receive a contact lens, which lens has an inner and an outer surface, the first and second baskets being oriented such that, in use, the respective inner surfaces of the contact lenses are remote from one another, wherein the reciprocating and vibrational motion generates bubbles in the cleaning liquid, which bubbles contribute to fluid turbulence at a surface of the contact lens, thereby assisting in the cleaning of the lens.

6. A method according to claim 5, characterized in that the cleaning solution has at least ten bubbles per 5 ml of liquid at any one time.

7. A method according to claim 5, wherein the cleaning liquid is a contact lens cleaning fluid or saline solution.

8. A method according to claim 5, wherein the reciprocating vibrational motion is applied for one minute, the device is turned off, and then the reciprocating-vibrational motion is applied for a further minute.

9. A device according to claim 2, wherein the means adapted to impart the reciprocating and vibrational motion comprises a motor (4), which motor (4) imparts a drive to an offset pin (10), which pin (10) is drivingly engaged in a slot (9) of a support arm (8), which support arm is positively connected to the lens holder (3), such that rotation of the off-set pin imparts said reciprocating and vibrational motion.

10. A device according to claim 3, wherein the frequency is 45 to 55 Hz.

11. A device according to claim 9, wherein the frequency is 45 to 55 Hz.

12. A method according to claim 6, wherein the cleaning liquid is a contact lens cleaning fluid or saline solution.

13. A method according to claim 6, wherein the reciprocating vibrational motion is applied for one minute, the device is turned off, and then the reciprocating-vibrational motion is applied for a further minute.

14. A method according to claim 7, wherein the reciprocating vibrational motion is applied for one minute, the device is turned off, and then the reciprocating-vibrational motion is applied for a further minute.

15. A method according to claim 12, wherein the reciprocating vibrational motion is applied for one minute, the device is turned off, and then the reciprocating-vibrational motion is applied for a further minute.
